(19) United States
(12) Patent Application Publication
(10) Pub. No.: US 2025/0092258 A1
(43) Pub. Date: Mar. 20, 2025

(54) Additive-Modified Thermoplastic Elastomer Composition

(71) Applicant: Becton, Dickinson and Company, Franklin Lakes, NJ (US)

(72) Inventors: Wen Yuan, Mountain Lakes, NJ (US); Xianhong Feng, Kearny, NJ (US)

(21) Appl. No.: 18/294,178

(22) PCT Filed: Aug. 9, 2022
(86) PCT No.: PCT/US2022/039769

§ 371 Date: Feb. 1, 2024

Related U.S. Application Data
(60) Provisional application No. 63/230,979, filed on Aug. 9, 2021.

(51) Int. Cl. C08L 83/04

(52) U.S. Cl. CPC C08L 83/04; C08L 2203/02; C08L 2205/06; C08L 2207/04

(57) ABSTRACT

A ultra-high molecular weight siloxane polymer modified membrane for use in closed system transfer device membranes.

### CROSS-REFERENCE TO RELATED APPLICATION

The present application claims priority to U.S. Provisional Application No. 63/230,979, entitled “Additive-Modified Thermoplastic Elastomer Composition”, filed Aug. 9, 2021, the entire disclosure of which is hereby incorporated by reference, in its' entirety.

### BACKGROUND OF THE INVENTION

### Field of the Disclosure

The present disclosure relates generally to a thermoplastic elastomer composition for use in a membrane for a medical device.

### Description of the Related Art

Health care providers reconstituting, transporting, and administering hazardous drugs, such as cancer treatments, can put health care providers at risk of exposure to these medications and present a hazard in the health care environment. Unintentional chemotherapy exposure can affect the nervous system, impair the reproductive system, and bring an increased risk of developing blood cancers in the future. Some drugs must be dissolved or diluted before they are administered, which involves transferring a solvent from one container to a sealed vial containing the drug in powder or liquid form, by means of a needle. Drugs may be inadvertently released into the atmosphere in gas form or by way of aerosolization, during the withdrawal of the needle from the vial and while the needle is inside the vial if any pressure differential between the interior of the vial and the surrounding atmosphere exists. In order to reduce the risk of health care providers being exposed to toxic drugs, the transfer of these drugs is accomplished utilizing a closed system transfer device or system.

Closed system transfer devices or systems may utilize membranes to ensure the safe transfer of fluid between components. For example, a syringe adapter may include a membrane that contacts a membrane of a mating component, such as a patient connector, IV bag spike, or vial adapter. Elastomers are commonly used to create a fluid tight seal between parts that are moving against each other. Particularly, elastomers serve to create a fluid tight seal against penetrating needles. Thermoplastic elastomers (TPEs) have been used throughout the medical industry because they exhibit unique properties that allow them to be easily manufacturable, and easily optimized for specific functional performances. TPEs are similar to synthetic rubber elastomers (or called thermoset rubber) in that they are elastic; however, they do not rely on permanent crosslinked structure for the elastic properties. In turn, this allows for TPE properties to be optimized though formulation and compounding while also providing benefits such as better recyclability and manufacturing efficiency.

Current closed system transfer devices or systems may include a membrane, which may be formed from a thermosetting isoprene rubber, which is pierced by a needle of a syringe adapter. Membranes in closed system transfer devices are required to be resealable, and have appropriate attachment/detachment force, fragmentation, and membrane tackiness. Accordingly, the membrane is required to meet sealing and leakage requirements while also limiting membrane fragmentation, which can create small material particles when the needle pierces through the membrane that can pose a risk to a patient. A lubricating agent, such as silicone oil, can be applied to the needle surface and the membrane to minimize membrane fragmentation. The use of a lubricating agent on the surface of the needle and membrane, however, can affect leakage performance, fragmentation, and flow rate through the syringe adapter.

### SUMMARY OF THE INVENTION

In accordance with an embodiment of the present invention, an ultra-high molecular weight siloxane membrane comprises from 0.5% to 2.5% of a lubricant additive material.

In accordance with another embodiment of the present invention, an ultra-high molecular weight siloxane membrane comprises from 0.5% to 2% of a lubricant additive material.

In accordance with another embodiment of the present invention, an ultra-high molecular weight siloxane membrane comprises from 0.5% to 1% of a lubricant additive material.

In accordance with another embodiment of the present invention, an ultra-high molecular weight siloxane membrane comprises from 1% to 2.5% of a lubricant additive material.

In accordance with another embodiment of the present invention, an ultra-high molecular weight siloxane membrane comprises from 1% to 2% of a lubricant additive material.

In accordance with another embodiment of the present invention, an ultra-high molecular weight siloxane membrane comprises a second lubricant additive material.

In accordance with another embodiment of the present invention, the lubricant additive material is Dow Corning MB50-002 and/or Silaplast ES7722-DS.

In accordance with another embodiment, an ultra-high molecular weight siloxane membrane includes two lubricant additives at a concentration level of from 0.5% to 3%.

In accordance with another embodiment, an ultra-high molecular weight siloxane membrane is used in a medical device.

### BRIEF DESCRIPTION OF THE DRAWINGS

The above-mentioned and other features and advantages of this disclosure, and the manner of attaining them, will become more apparent and the disclosure itself will be better understood by reference to the following descriptions of aspects of the disclosure taken in conjunction with the accompanying drawings, wherein:

FIG. 1 is an illustration of a Compression Set B (CSB) setup in accordance with a conventional testing procedure.

FIG. 2 is an illustration of an SEM image of a sample top surface in accordance with an embodiment of the present invention.

FIG. 3 is a graphical representation of a leakage plot for injector membranes and connector membranes in accordance with an embodiment of the present invention.

Corresponding reference characters indicate corresponding parts throughout the several views. The exemplifications set out herein illustrate exemplary aspects of the disclosure, and such exemplifications are not to be construed as limiting the scope of the disclosure in any manner.

### DETAILED DESCRIPTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

One potential solution to the issues identified above is to apply lubricant oil, or other low surface energy polymer such as fluoropolymer or silicone emulsion, directly on the surface of the membrane by spray coating or soaking techniques to lower the friction between needle and membrane. With a limitation on migrating polymer intrinsically, this method will not be applicable for a thicker membrane and not be suitable for multi-penetration usage. Another solution is to use thermoplastic elastomer.

In one aspect or embodiment of the present invention, a very small amount of lubricating agent, such as ultra-high molecular weight siloxane polymer, is compounded into a thermoplastic elastomer formulation for use in a medical device membrane. The resulted material improves intrinsic lubricity from siloxane polymer. In addition, such a high molecular weight and pre-compounded olefin based masterbatch will help blending uniformly and has less tendency of migrating to the surface. The molded parts with this new compounding will improve fragmentation, lower membrane tackiness and attachment force while has negligible scarifying on leakage performance. This additive also can be utilized for other TPE related applications.

The membrane may be utilized in any component of a closed system transfer device or system, such as a syringe adapter, patient connector, vial adapter, IV bag spike, etc. The membrane may be utilized with the syringe adapter shown and described in United States Patent Application Publication No. 2015/0297454, which is hereby incorporated by reference in its entirety. The membrane may also be utilized in other medical device components and, more specifically, medical device components where the membrane is punctured by a needle.

Thermoplastic elastomer (TPE) provides similar properties to conventional rubber materials such as thermoset rubber and silicone rubber. TPE is crosslinked by polymer chain physical interaction not via covalent bonding, thus it is recyclable and easier to process compared to thermoset rubber and silicone. Extruded and molded TPE articles are widely used as crucial components in medical device applications, such as septum, stopper, resealable membrane and tubing, which generally require high elasticity, flexibility and great stability.

Table 1 identifies the various formulations. Compounding extrusion was performed to produce all the formulations in Table 1, including two lubricant additives at various concentration level from 0.5% to 3%. The resulted resins were molded to standard ASTM sheets from which tensile, tear, compression set, and abrasion testing samples were produced. The resulting materials were evaluated to understand how additive concentration in TPE impact the material's mechanical properties, which potentially will later reflect on product performance.

TABLE 1
Formulations

| | Parts/PHR | | | | | | |
|---|---|---|---|---|---|---|---|
| | | | | Dow | | | |
| | | | | Corning ® | Silaplast | | |
| Formula- | | | | MB | ES7722- | Slip | Anti- |
| tion | SEBS | PP | Oil | 50-002 | DS | agent | oxidant |
| Base | 100 | 25 | 125 | — | — | 1 | 1 |
| M1 | 100 | 25 | 125 | 1.5 | — | 1 | 1 |
| M2 | 100 | 25 | 125 | 2.5 | — | 1 | 1 |
| M3 | 100 | 25 | 125 | 5 | — | 1 | 1 |
| M4 | 100 | 25 | 125 | 7.5 | — | 1 | 1 |
| S1 | 100 | 25 | 125 | — | 2.5 | 1 | 1 |
| S2 | 100 | 25 | 125 | — | 5 | 1 | 1 |

One of lubricant additives, Dow Corning MB50-002, has been identified for compounding with a TPE formulation at various loadings from 0.5% to 2% shown in Table 2. The resultant lubricant additive modified TPEs were processed via injection molding and the molded parts were assembled for product testing. The assembled closed system transfer medical device was then tested on product performance including leakage, fragmentation, attachment force and detachment force.

TABLE 2
List of TPE formulation containing silicone additives
for injection molding and product testing

| | Formulation |
|---|---|
| 1 | Neat TPE (Mediprene) |
| 2 | TPE + 0.5% MB50-002 |
| 3 | TPE + 1% MB50-002 |
| 4 | TPE + 2% MB50-002 |

All the extruded compounds with siloxane fillers were then characterized using mechanical methods. Tensile testing was performed using samples prepared from the molded materials in accordance to ASTM D412. Five samples were run for each sample; from the data, tensile strength was calculated and reported in Table 3. It is found MB50 consistently enhance tensile strength up to 25% while Silaplast additive maintained the same performance.

TABLE 3
Tensile strength, Tear strength and Abrasion notch depth results
for formulations M1 through S2 and the base material.

| | Tensile | Tear | Abrasion |
|---|---|---|---|
| | Strength | Strength/ | Notch |
| Formulation | at Break/Mpa | N/m | Depth/um |
| Base | 4.2 | 16682 | 65.6 |
| M1 | 5.6 | 21087 | 80.4 |
| M2 | 5.2 | 20844 | 54.6 |
| M3 | 5.6 | 20544 | 47.3 |
| M4 | 4 | 18490 | 60.2 |
| S1 | 4.2 | 22182 | 60 |
| S2 | 4.6 | 20775 | 82.2 |

Tear strength was performed using ASTM D624 tear strength samples prepared from the molded materials. Four samples were run for each of the materials; from the results of the test, tear strength was calculated and is presented in Table 3. Both MB50 and Silaplast improved tear strength with the silicone additive loading no more than 5 phr.

Abrasion testing was performed to understand the notch depth after exposure to a repetitive surface abrasion. Briefly, a 50 g weight was used to cyclically scratch the surface of all the formulations produced; 1120 total cycles were run, with the tip of the abrasion machine being cleaned with isopropyl alcohol and a kimwipe every 280 cycles. The materials were allowed to rest for 24 hours and were then run under the profilometer to measure the depth of the abrasion. Table 3 outlines the abrasion depths obtained from the software. It is clearly shown that a higher loading of MB50 will reduce notch depth over 25%. This property improvement is correlated with improved fragmentation and attachment force performance of new membrane formulations.

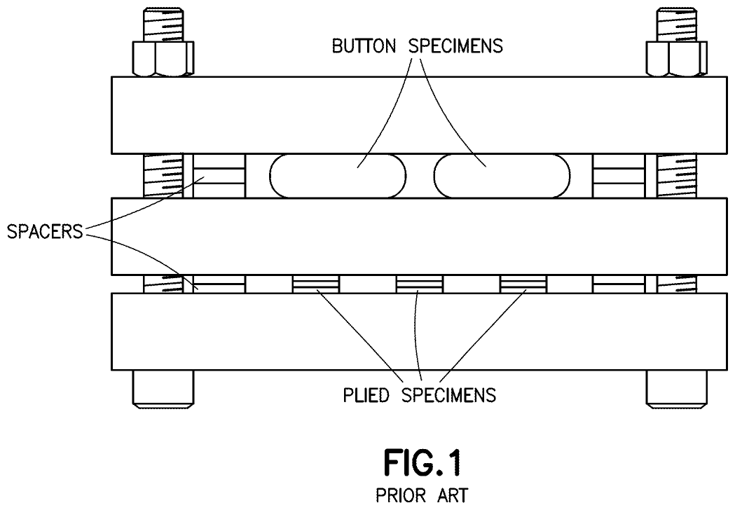

Compression Set B (CSB) was measured using a CSB setup, as shown in FIG. 1 in accordance to ASTM D395. Polymeric samples are placed in between sandwiched plates of metal with a metal spacer of known thickness in between. After the materials are placed in the setup and the contraption tightened, the material is allowed to rest for a defined amount of time; afterwards, the contraption is disassembled and materials allowed to rest briefly before measurements are taken. From a formed sheet, twelve 10 mm-wide discs were cut, and four of each stacked on each other to produce samples of between 6.5-7.5 mm thickness. The thickness of the samples were measured and then placed in a setup with 4.8 mm-thick metal washers; the materials were then compressed to 4.8 mm and left to rest for 22 hours at room temperature. After releasing them, they were allowed to rest for 30 minutes before their new thickness was measured. Using Equation (1), average CSB and their standard deviations were calculated across all samples.

$$\begin{matrix}Equation 1 & \\ CSB = 100 * \frac{{T}_{initial} - {T}_{final}}{{T}_{initial} - {T}_{spacer}} & ( 1 )\end{matrix}$$

Tᵢₙᵢₜᵢₐₗ is the initial thickness of the four stacked discs, T_{final} is their thickness after 22 hrs of compression and 30 minutes of resting, and T_{spacer}—is the thickness of the washers used as the spacers (4.8 mm). The results of this calculation are identified in Table 4.

TABLE 4
Average Compression Set B of formulations
with MB50 and Silaplast.

| | Filler, | | Average | St Dev. |
|---|---|---|---|---|
| Formulation | Parts | n | CSB | CSB |
| Base | n.a. | 3 | 8.0 | 1.7 |
| M3 | MB, 5 | 3 | 10.9 | 2.8 |
| S2 | SP, 5 | 3 | 15.0 | 1.4 |

For base formulation, M3 and S2 shown in Table 4, a 10 mm flat material disc was used for testing. For Mediprene and other MB50 contained Mediprene formulation, a molded connector membrane was used for testing shown in the Table 5.

TABLE 5
Average Compression Set B of Neat Mediprene
and silicone contained Mediprene formulation.

| | | Average | St Dev. |
|---|---|---|---|
| Formulation | n | CSB | CSB |
| Neat TPE (Mediprene) | 3 | 9.2 | 1.5 |
| TPE + 0.5% MB50-002 | 3 | 9.0 | 0.2 |
| TPE + 1% MB50-002 | 3 | 8.6 | 1.5 |
| TPE + 2% MB50-002 | 3 | 9.2 | 1.4 |

With the addition of 5 phr (˜2 wt %) of lubricant additive or more, the compression set went higher on both MB50 and Silaplast as shown in Table 3. Once the loadings of MB50 in TPE formulation reduced equal to or less than 2 wt %, the compression set remained in the same range shown in Table 5.

Coefficient of friction (COF) and contact angle have also been measured with respect to the material surface properties, as shown in Table 6. With the addition of lubricant additive, both static and kinetic COF were significantly reduced up to about 50%. Contact angle results indicated Silaplast ES7722-DS could reduce surface energy while Dow Corning® MB 50-002 maintained almost the same. This improved property also correlates well with improved tackiness, fragmentation and attachment force performance of the new membrane formulations.

TABLE 6
Coefficient of Friction and contact angle measurement
with addition of silicone additives

| | COF- | COF- | Contact |
|---|---|---|---|
| TPE | Static | Kinetic | Angle |
| Base | 1.194 | 1.145 | 96.42 |
| M2 | 0.722 | 0.696 | 92.01 |
| M3 | 0.751 | 0.702 | 94.36 |
| S1 | 0.688 | 0.666 | 100.70 |
| S2 | 0.863 | 0.784 | 104.67 |

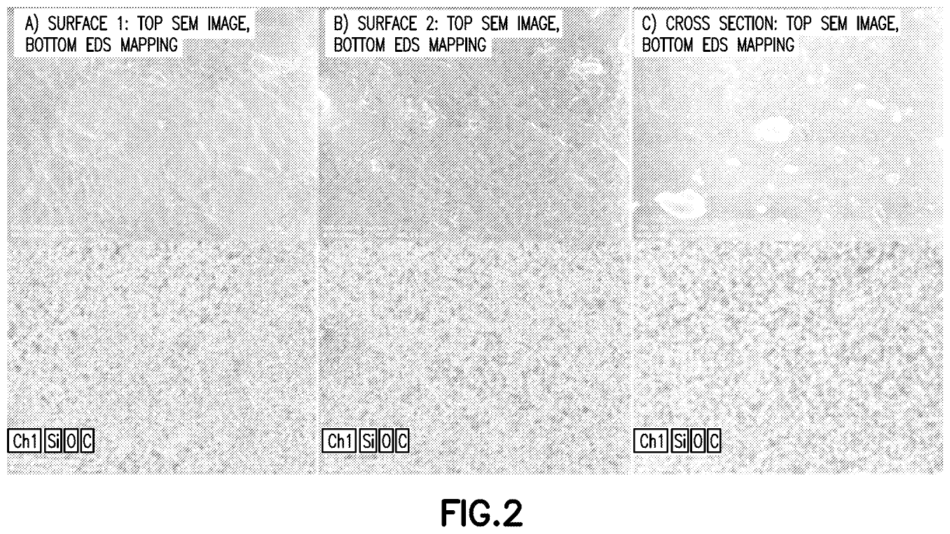

In order to understand the silicone additive's distribution across the TPE sample, one of TPE formulation (M3) has been selected to determine material composition distribution by using SEM. Carbon, oxygen and silicon on top surface, bottom surface and cross section of membrane have been mapped in the EDS mode of SEM, as shown in FIG. 2. With continued reference to FIG. 2, in which (a) reflects a top surface of SEM and EDS mapping of silicone additive contained TPE, (b) reflects a bottom surface of SEM and EDS mapping of silicone additive contained TPE, and (c) reflects a cross-section of SEM and EDS mapping of silicone additive contained TPE.

As shown in Table 7, silicon atom % remained the same order of magnitude with a value from 0.1% to 0.18% among two surfaces (top and bottom) and the cross section. This illustrates uniform distribution of lubricant additives of the inventive membrane formulation. This characteristic illustrates the improved lubricity of the membrane both internally and externally.

TABLE 7
Relative element percentage on top surface, bottom surface,
and cross section of silicone additive contained TPE sample.

| | Element | Mass % | Atom % |
|---|---|---|---|
| Top Surface | Carbon | 91.3 | 93.39 |
| | Oxygen | 8.48 | 6.51 |
| | Silicon | 0.23 | 0.1 |
| Bottom Surface | Carbon | 94.49 | 95.9 |
| | Oxygen | 5.21 | 3.97 |
| | Silicon | 0.3 | 0.13 |
| Cross section | Carbon | 96.97 | 97.84 |
| | Oxygen | 2.62 | 1.99 |
| | Silicon | 0.41 | 0.18 |

The tackiness performance of new membrane formulation was measured by membrane-to-membrane separation force where two membranes with flat surfaces were compressed together and separated by Instron to simulate worst case condition of storage/shipping/assembly process. As shown in the Table 8, the tackiness performance of new membrane formulation improved with addition of Ultra-high molecular weight siloxane polymer such as DOW CORNING® MB50-002, etc. This will reduce the membrane scrape rate, increase manufacturing efficiency and reduce the overall cost of products.

TABLE 8
Tackiness performance of new membrane formulation

| | Tackiness |
|---|---|
| Formulation | (Separation Force)/N |
| Mediprene | 0.38 |
| Mediprene + 0.5% additive | 0.27 |
| Mediprene + 1% additive | 0.19 |
| Mediprene + 2% additive | 0.23 |

As shown in Table 9, without silicone oil on the needle surface and in the membrane pocket, the fragmentation performance of new membrane pass acceptance requirement and improved significantly with addition of Ultra-high molecular weight siloxane polymer additives. The intrinsic lubricity from the new additive modified new membrane formulation significantly reduced the friction between needle and membrane which resulted in reduced fragments as well as safety performance of CSTD products.

TABLE 9
Fragmentation performance of new membrane formulation without
silicone oil on the needle surface and in the membrane pocket

| | Average fragmentation |
|---|---|
| Formulation | score |
| Mediprene | 35.4 |
| Mediprene + 0.5% additive | 22.8 |
| Mediprene + 1% additive | 17.6 |
| Mediprene + 2% additive | 18.4 |

As shown in Table 10, without silicone oil on the needle surface and in the membrane pocket, the attachment performance of new membrane pass acceptance requirement and improved significantly with addition of Ultra-high molecular weight siloxane polymer additives. The intrinsic lubricity from the Ultra-high molecular weight siloxane polymer modified new membrane formulation significantly reduce the friction between needle and membrane which resulted in reduced attachment force as well as safety performance of closed system transfer device products.

TABLE 10
Attachment performance of new membrane formulation without silicone
oil on the needle surface and in the membrane pocket

| | Average attachment |
|---|---|
| Formulation | force/N |
| Mediprene | 54.9 |
| Mediprene + 0.5% additive | 50.6 |
| Mediprene + 1% additive | 50.1 |
| Mediprene + 2% additive | 49.1 |

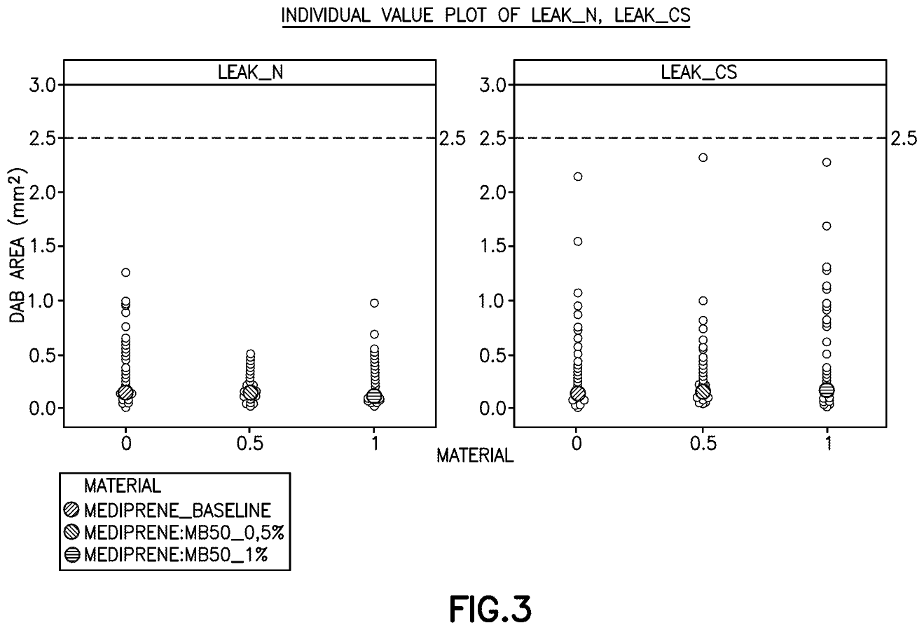

As shown in FIG. 3, without silicone oil on the needle surface and in the membrane pocket, the new membrane formulation with addition of <1% Ultra-high molecular weight siloxane polymer such as DOW CORNING® MB50-002 improved the leakage performance of membrane. With continued reference to FIG. 3, it is noted that N is the injection membrane and CS is the connector membrane. The intrinsic lubricity from the lubricant additive modified new membrane formulation significantly reduced the friction between needle and membrane which resulted in less coring and damage of membrane during the needle penetration process thus improve the sealing performance of closed system transfer device products.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. To the extent possible, one or more features of any aspect or embodiment discussed above can be combined with one or more features of any other aspect or embodiment.

1. An ultra-high molecular weight siloxane membrane comprising:
from 0.5% to 2.5% of a lubricant additive material.

2. The membrane of claim 1, wherein the ultra-high molecular weight siloxane membrane comprises from 0.5% to 2% of a lubricant additive material.

3. The membrane of claim 1, wherein the ultra-high molecular weight siloxane membrane comprises from 0.5% to 1% of a lubricant additive material.

4. The membrane of claim 1, wherein the ultra-high molecular weight siloxane membrane comprises from 1% to 2.5% of a lubricant additive material.

5. The membrane of claim 1, wherein the ultra-high molecular weight siloxane membrane comprises from 1% to 2% of a lubricant additive material.

6. The membrane of claim 1, further comprising a second lubricant additive material.

7. The membrane of claim 1, wherein the lubricant additive material is Dow Corning MB50-002.

8. The membrane of claim 1, including two lubricant additives at a concentration level of from 0.5% to 3%.

9. The membrane of claim 1, for use in a medical device.

10. The membrane of claim 1, wherein the lubricant additive material is Silaplast ES7722 DS.
